(19) United States
(12) Patent Application Publication
(10) Pub. No.: US 2010/0089405 A1
(43) Pub. Date: Apr. 15, 2010

(54) COMBINATION TRACHEAL HOOK AND SURGICAL BLADE

(75) Inventors: Ross Johnson, Anderson, SC (US); Richard A. Hester, Greenville, SC (US)

Correspondence Address: CAHN & SAMUELS LLP, 1100 17th STREET NW, SUITE 401, WASHINGTON, DC, 20036, US

(73) Assignee: Tactical Medical Solutions, Inc., Anderson, SC (US)

(21) Appl. No.: 12/418,278

(22) Filed: Apr. 3, 2009

Related U.S. Application Data
(60) Provisional application No. 61/042,658, filed on Apr. 4, 2008.

(51) Int. Cl. A61M 16/00; A61B 17/3211

(52) U.S. Cl. 128/207.29

(57) ABSTRACT

An embodiment of the invention provides a surgical tool including a handle having a channel, a tracheal hook connected to the handle, and a scalpel positioned in the channel of the handle. The tracheal hook and/or the scalpel are removably attached to the handle. The channel includes at least one first fastener (e.g., a recess); and, the scalpel includes at least one complementary second fastener (e.g., a protrusion) for engaging the first fastener. The scalpel and/or handle include a light source. The tracheal hook and/or the handle are formed from translucent material. In addition, a wire is provided having a first end connected to the handle and a second end connected to the tracheal hook. The surgical tool further includes at least one cap having a first open end, a second open end, and at least one lateral fenestration.

### I. CROSS-REFERENCE TO RELATED APPLICATIONS

This application claims the benefit of U.S. Provisional Patent Application Ser. No. 61/042,658 filed Apr. 4, 2008, incorporated herein by reference.

### II. FIELD OF THE INVENTION

The present invention relates to surgical instruments for use in performing emergency surgical airway or cricothyrotomies/tracheostomies, and more particularly to a combination tracheal hook and scalpel which are separable from each other.

### III. BACKGROUND OF THE INVENTION

When the laryngeal opening of a patient becomes obstructed, blocking the flow of air to the lungs, an emergency surgical airway must be created to relieve the obstruction. In addition, when the laryngeal opening cannot be visualized (for normal intubation) because of the presence of blood or vomit, an emergency surgical airway is often necessary to save the patient. This procedure is commonly referred to as a cricothyrotomy or tracheostomy. Techniques for performing an emergency surgical cricothyrotomy/tracheostomy vary, but generally, the technique is a multi-step procedure that requires the use of a scalpel for creating an incision, a dilation instrument, and a tracheal hook for stabilizing the larynx. The group of surgical instruments used to create the surgical airway is often collectively included in a surgical airway kit or surgical airway instrument tray.

Although such kits are effective at providing the tools necessary to perform a cricothyrotomy/tracheostomy, currently available kits have several problems. One problem is the amount of time required to gather and/or access multiple tools which can delay the establishment of the airway to the patient's detriment. Because surgical cricothyrotomy is typically performed during an emergency, time is of the essence and every second counts. Rapid creation of the emergency airway increases the likelihood of saving the patient's life.

Another problem with currently available surgical kits is that it is often difficult to maintain control of multiple instruments in an austere environment. A care giver is frequently called upon to establish an emergency airway in the patient at the scene of an accident, on the battlefield or in an emergency room setting. Rarely can the caregiver rely on an assistant to locate instruments and insure that those instruments are in condition for use.

A number of devices have been developed in an attempt to combine several surgical instruments into one device which can be used to open an emergency airway. While a combined device eliminates some of the above-mentioned problems, combined devices that are presently available restrict the care giver to using one instrument at a time. This creates a significant problem if the initial incision is too small. For example, the tracheal hook on a combined device must be removed from the trachea so that the attached scalpel can be used to enlarge the opening. When this is done, the care giver loses control of the initial hole and must either start over or spend time searching for the hole costing valuable seconds or even minutes, particularly with an inexperienced individual.

### IV. SUMMARY OF THE INVENTION

An embodiment of the invention provides a surgical tool including a handle having a channel, a tracheal hook connected to the handle, and a scalpel positioned in the channel of the handle. The tracheal hook and/or the scalpel are removably attached to the handle. The channel includes at least one first fastener (e.g., a recess); and, the scalpel includes at least one complementary second fastener (e.g., a protrusion) for engaging the first fastener.

In one embodiment, the scalpel includes a light source positioned to illuminate an area proximate the scalpel. The handle can also include a light source positioned to illuminate an area proximate the tracheal hook. The tracheal hook and/or the handle are formed from transparent or translucent material. In addition, a wire is provided having a first end connected to the handle and a second end connected to the tracheal hook. The surgical tool further includes at least one cap having a first open end, a second open end, and at least one lateral fenestration.

In another embodiment of the invention, the surgical tool comprises a tracheal hook that includes a sheath that defines a channel. A scalpel is positioned in the channel of the sheath, such that the scalpel is removably attached to the tracheal hook. In accordance with an aspect of the invention, the tracheal hook has at least one first fastener; and, the scalpel has at least one complementary second fastener for engaging the first fastener. Thus, the scalpel is removably attached to the tracheal hook. In one embodiment, the first fastener includes a T-tab; and, the second fastener includes a T-slot. In another embodiment, the first fastener includes a T-slot; and, the second fastener includes a T-tab.

An embodiment of the invention provides a method for utilizing a combination tracheal hook and scalpel tool. An airway is created proximate a patient's trachea using a scalpel of the combination tracheal hook and scalpel tool. A tracheal hook of the combination tracheal hook and scalpel tool is inserted into the airway. The scalpel is removed from the combination tracheal hook and scalpel tool; and, the airway is modified using the scalpel while the tracheal hook is positioned in the airway. In one embodiment, the scalpel is removed after the tracheal hook is inserted into the airway. The tracheal hook can also be removed from the combination tracheal hook and scalpel tool. Moreover, an open-ended cap is inserted into the airway. The method also illuminates the area proximate the airway by activating a light source on the combination tracheal hook and scalpel tool.

In the following enabling description, reference is made to the accompanying drawings which are shown by way of illustration of the specific embodiments in which the invention may be practiced. In the following embodiments the apparatus and methods should become evident to a person of ordinary skill in the art and in sufficient detail to enable those skilled in the art to practice the invention. It is to be understood that other embodiments may be used and that structural changes based on presently known structural and/or functional equivalents may be made without departing from the scope of the invention.

### V. BRIEF DESCRIPTION OF THE DRAWINGS

The present invention is described with reference to the accompanying drawings, wherein:

FIG. 1A illustrates a surgical tool according to an embodiment of the invention;

FIG. 1B illustrates a surgical tool having a rectangular shaped finger guard according to an embodiment of the invention;

FIG. 1C illustrates a side view of the surgical tool of FIG. 1B;

FIG. 1D illustrates a removable scalpel according to an embodiment of the invention;

FIG. 1E illustrates a side view of the removable scalpel of FIG. 1D;

FIG. 1F illustrates a top view of the removable scalpel of FIG. 1D;

FIG. 1G illustrates a bottom view of a removable hook according to an embodiment of the invention;

FIG. 2 illustrates a channel according to an embodiment of the invention;

FIG. 3 illustrates a handle having an enlarged opening according to an embodiment of the invention;

FIG. 4 illustrates a scalpel according to an embodiment of the invention;

FIG. 5 illustrates a reinforcement ridge according to an embodiment of the invention;

FIG. 6 illustrates a light source according to an embodiment of the invention;

FIG. 7 illustrates an open-ended cap according to an embodiment of the invention;

FIG. 8A illustrates a surgical tool having a sheath according to an embodiment of the invention;

FIG. 8B illustrates a side view of the surgical tool of FIG. 8A;

FIG. 9A illustrates a hook having a finger guard according to an embodiment of the invention;

FIG. 9B illustrates a scalpel according to an embodiment of the invention;

FIG. 9C illustrates a hook according to an embodiment of the invention;

FIG. 9D illustrates a tool having a hook and a scalpel according to an embodiment of the invention;

FIG. 9E illustrates a perspective view of a T-tab fastener according to an embodiment of the invention;

FIG. 9F illustrates a side view of a T-tab fastener illustrated in FIG. 9E;

FIG. 9G illustrates a front view of a T-slot fastener according to an embodiment of the invention; and

FIG. 10 illustrates a flow diagram of a method for creating a surgical airway according to an embodiment of the invention.

### VI. DETAILED DESCRIPTION OF THE DRAWINGS

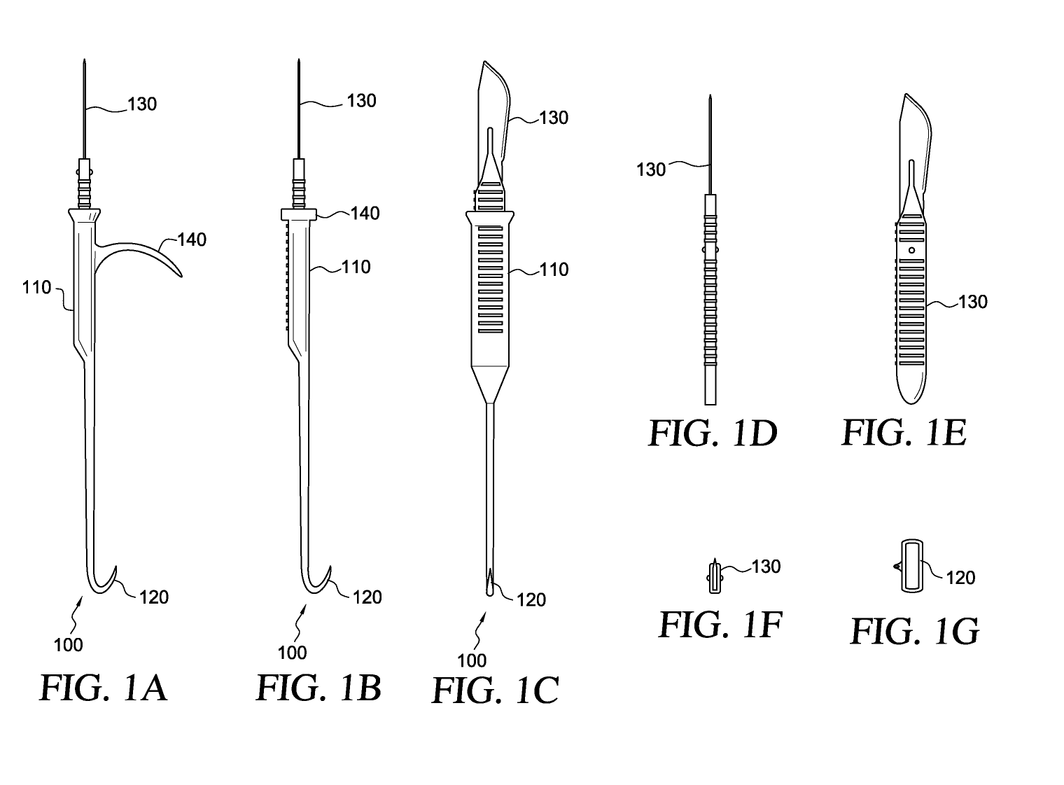

As illustrated in FIGS. 1A-1G, an embodiment of the invention provides a combination hook and scalpel tool 100 (also referred to herein as the “surgical tool”), including a handle 110, a hook 120, and a scalpel 130. The handle 110 can be formed from plastic, composite, and/or metal, and includes a smooth (FIG. 1A) or textured (e.g., gripping ridges; FIGS. 1B-1C) gripping surface. The handle 110 includes a first end and a second end opposite the first end. Either or both of the hook 120 and the scalpel 130 are removably attached to the handle 110. For example, in one embodiment, the hook 120 is permanently affixed or integrally formed to the first end of the handle 110; and, the scalpel 130 is removably attached to the second end of the handle 110. In another embodiment, the hook 120 is removably attached to the first end of the handle 110; and, the scalpel 130 is permanently affixed or integrally formed with the second end of the handle 110. In yet another embodiment, both the hook 120 and the scalpel 130 are connected to the same end of the handle 110, e.g., the hook 120 and the scalpel 130 are both connected to the first end of the handle 110.

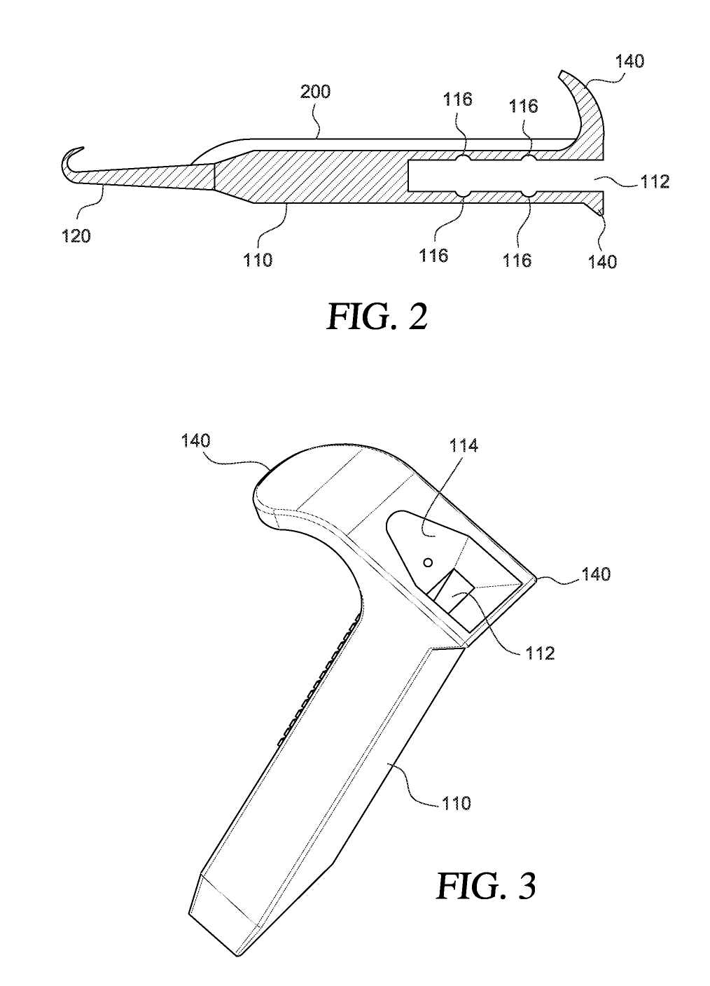

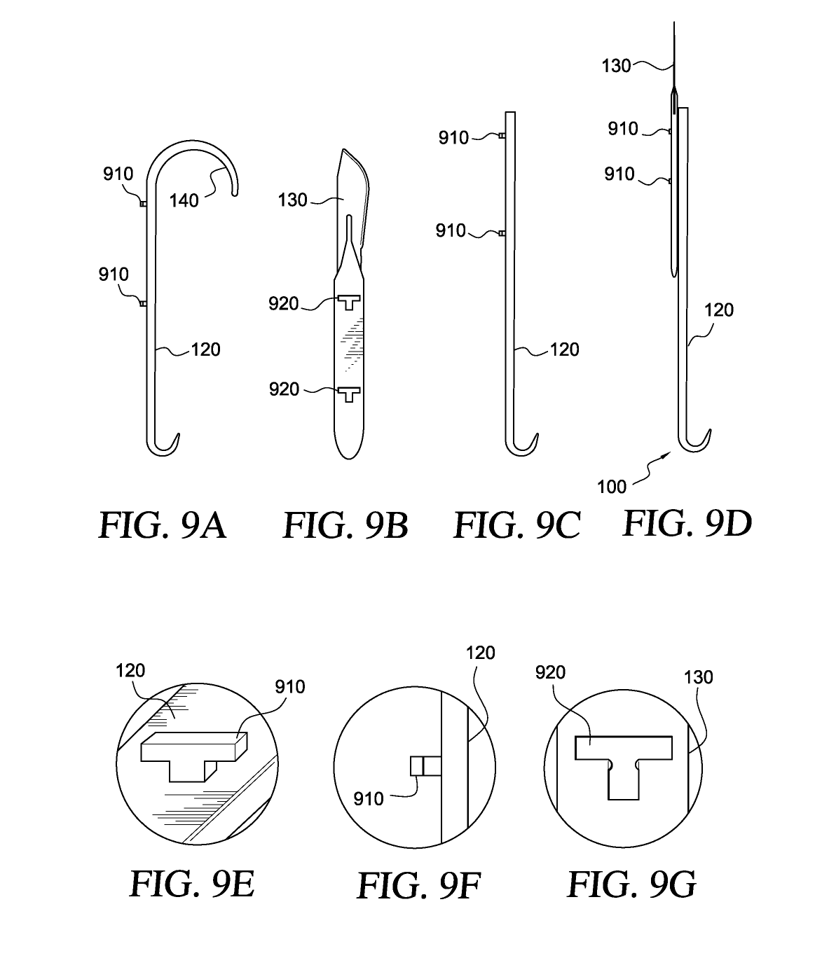

In at least one embodiment, the second end of the handle 110 is beveled to assist insertion of the scalpel 130 and prevent the user from cutting or poking herself with the blade. In another embodiment, the handle 110 includes a channel 112 for receiving the scalpel 130 (FIGS. 2-3). In accordance with an aspect of the length of the channel 112 may be shorter than the length of the handle 110. As such, a portion of the scalpel 130 protrudes from the handle 110 when inserted into the channel 112 facilitating ready removal of the scalpel 130 from the handle 110. In one embodiment, the channel 112 includes an enlarged opening 114 having an inclined ramp for receiving and guiding the scalpel 130 into the channel 112 (FIG. 3). The channel 112 may be dimensioned such that the scalpel 130 may be positioned within the handle 110 either blade side down (FIG. 9) or blade side up (FIG. 1A).

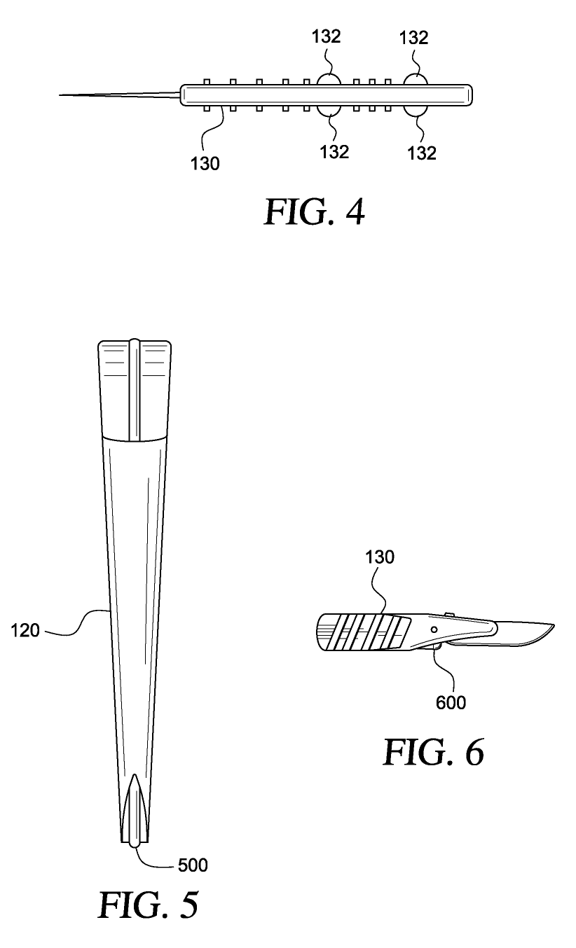

In keeping with the invention, as illustrated in FIG. 2, the channel 112 includes first fasteners 116 for engaging and locking with complementary second fasteners 132 on the scalpel 130. In at least one embodiment of the invention, the first fasteners 116 include recesses, or indentations. Although the recesses are shown to have concave or semicircle-shaped cross-sections, it is recognized that the recesses could have other shapes (e.g., square, rectangular, triangular). In one embodiment, as illustrated in FIG. 4, the second fasteners 132 of the scalpel 130 engage first fasteners 116 in a locking relationship. For example, the second fasteners 132 include protrusions that are adapted to interlock with the recesses in the channel 112. Although the protrusions are shown to have convex or semicircle-shaped cross-sections, it is recognized that the protrusions could have other shapes (e.g., square, rectangular, triangular). The protrusions may be disengaged from the recesses by pulling the scalpel 130 out of the channel 112.

The first fasteners 116 may be located at varying depths within the channel 112 to accept scalpels of different lengths or to adjust the exposed length of a single scalpel. It is recognized that other fastening means could be utilized to connect the scalpel 130 to the handle 110, such as, for example, bolts, screws, rivets, clamps, clasps, clips, pins, and/or adhesive. In addition, the first fasteners 116 may be disposed in the handle of the scalpel 130 and the second fasteners 132 may be formed in the channel 112.

In at least one embodiment, the handle 110 includes one or more finger guards 140 located proximate the opening 114 comprising tabs that extend generally outward from the handle 110. The finger guards 140 provide a gripping surface when the tool 100 is in use. Although the finger guards 140 are illustrated as arcuate (FIG. 1A), rectangular (FIG. 1B), and triangular (FIG. 3), it is recognized that the finger guards 140 could embody other shapes that prevent slippage of a user's hand.

The hook 120 (also referred to herein as the “tracheal hook”) can be formed from plastic, composite, and/or metal. In some embodiments, the hook 120 may be 4-6 inches long and tapered to a point or truncated. In one embodiment, as illustrated in FIG. 5, the hook 120 includes a reinforcement ridge 500 for increased strength and rigidity. As discussed above, the hook 120 can be permanently affixed to, integrally formed with, or removably attached to the handle 110. For example, the hook 120 could be connected to the handle 110 using bolts, screws, rivets, clamps, clasps, clips, pins, and/or adhesive.

In accordance with an aspect of the invention, the scalpel 130 (also referred to herein as the “surgical blade”) is preferably formed from metal, has a length of 3-4 inches, and includes a textured (e.g., ridges) or smooth handle to provide a gripping surface when detached from the handle 110. As discussed above, the scalpel 130 can be permanently affixed to, integrally formed with, or removably attached to the handle 110. For example, as illustrated in FIG. 4, the scalpel 130 includes protrusions (i.e., second fasteners 132) for engaging recesses (i.e., first fasteners 116) in the channel 112. The protrusions are preferably formed from rubber; although, it is recognized that the protrusions could be formed from other materials, such as, for exemplary purposes only, metal, plastic, or other composites.

While treating patients during emergency situations, it is not uncommon for a caregiver to misuse surgical instruments, especially if pressured under stressful and inhospitable conditions. If too much force is applied to the instrument, the handle can fracture and/or the hook can break off inside of a patient's trachea. In an embodiment of the invention, as illustrated in FIG. 2, the tool 100 includes a connector 200 (e.g., wire, string, cord, thread, twine, rope, lanyard, yarn, line) for tethering the hook 120 to the handle 110. Specifically, the connector 200 has a first end attached to the handle 110 (e.g., the finger guard 140) and a second end attached to the hook 120. Thus, if the handle breaks while the hook 120 is positioned within the tracheal airway of a patient, the connector 200 may be used to remove the hook 120.

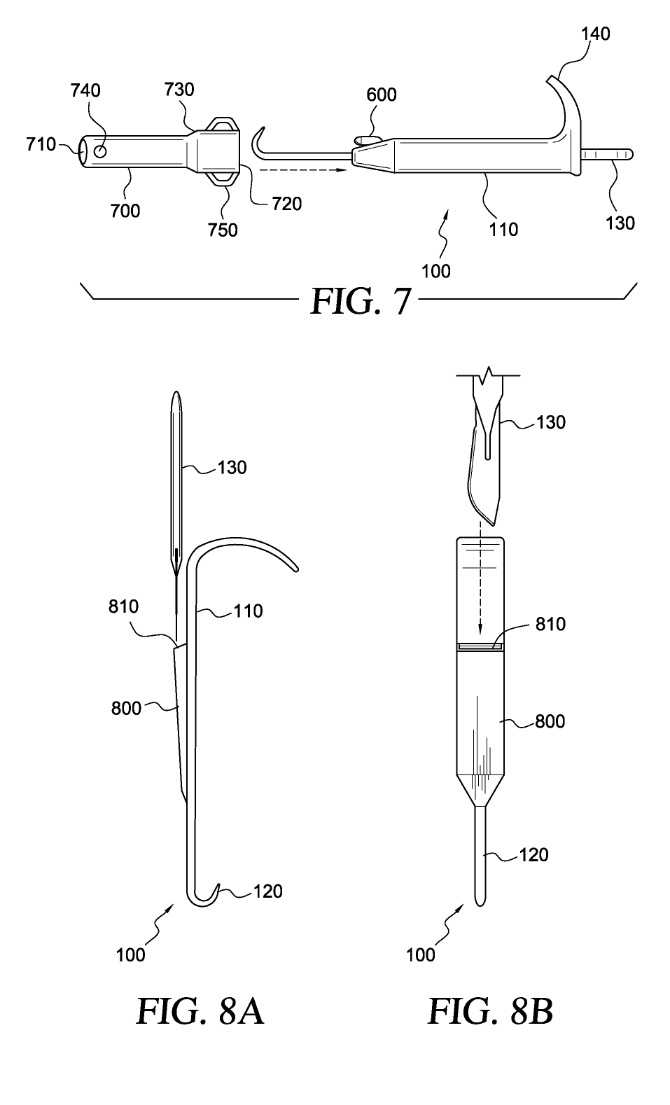

In at least one embodiment, as depicted for example in FIG. 6, the handle 110, hook 120, and/or the scalpel 130 includes a light source 600 (e.g., LED) to increase visibility while utilizing the tool 100. The light source 600 may be positioned on the scalpel 130 such that light emitting from the light source 600 illuminates the area to be cut by the scalpel 130. In another embodiment, as illustrated in FIG. 7, the light source 600 is positioned on the handle 110, such that light emitting from the light source 600 is directed toward the hook 120. In one embodiment, the hook 120 is formed from transparent or translucent material, such that light from the light source 600 illuminates the tracheal airway when the hook 120 is inserted into the patient.

In at least one embodiment of the invention, the tool 100 includes one or more removable caps 700 for covering the hook 120 and/or the scalpel 130. The cap 700 includes a first open end 710 and a second open end 720. The cap 700 is not only useful as a means to protect the hook 120; the first open end 710 of the cap 700 may be inserted into a patient's trachea to maintain an open airway during surgical procedures. The second open end 720 has a larger circumference than the first open end 710 and includes a cuff 730 for preventing the cap from slipping too far into the patient's trachea. The cap 700 also includes one or more lateral fenestrations 740 to provide additional air passages if one or both of the open ends 710, 720 are blocked by tissue or other obstructions. Furthermore, the cap 700 includes one or more bracket loops 750 disposed on the cuff 730 for receiving a tracheal tube support mechanism (e.g., straps, rope, cords, twine) therein.

In some embodiments of the invention, the tool 100 lacks a handle 110. For example, in an embodiment illustrated in FIGS. 8A-8B, the hook 120 includes a sheath 800 having a channel 810 for receiving and retaining the scalpel 130 therein. Preferably, the sheath 800 is formed from plastic. However, it is recognized that the sheath 800 could be formed from other materials, such as, for exemplary purposes only, metal, leather, rubber, or the sheath could be integrally formed to the handle 110.

In an embodiment depicted in FIGS. 9A-9G, the hook 120 has T-tab fasteners 910 (FIG. 9A); and, the scalpel has T-slot fasteners 920 (FIG. 9B). FIG. 9C illustrates the hook 120 according to another embodiment of the invention. Thus, as illustrated in FIG. 9D, the hook 120 can be removably attached to the scalpel 130 by inserting and locking the T-tab fasteners 910 within the T-slot fasteners 920. FIG. 9E illustrates a perspective view of a T-tab fastener 910; and, FIG. 9F illustrates a side view of the T-tab fastener 910. FIG. 9G illustrates a front view of a T-slot fastener 920.

In another embodiment, the hook 120 includes the T-slot fasteners 920 (FIG. 9G); and, the scalpel 130 includes the T-tab fasteners 910 (FIGS. 9E and 9F). In accordance with the invention, other fasteners could be utilized, such as, for example, brackets, clips, clasps, other shaped tab-and-slot arrangements, and/or the hook 120 can be otherwise slidably and removably engaged with the scalpel 130.

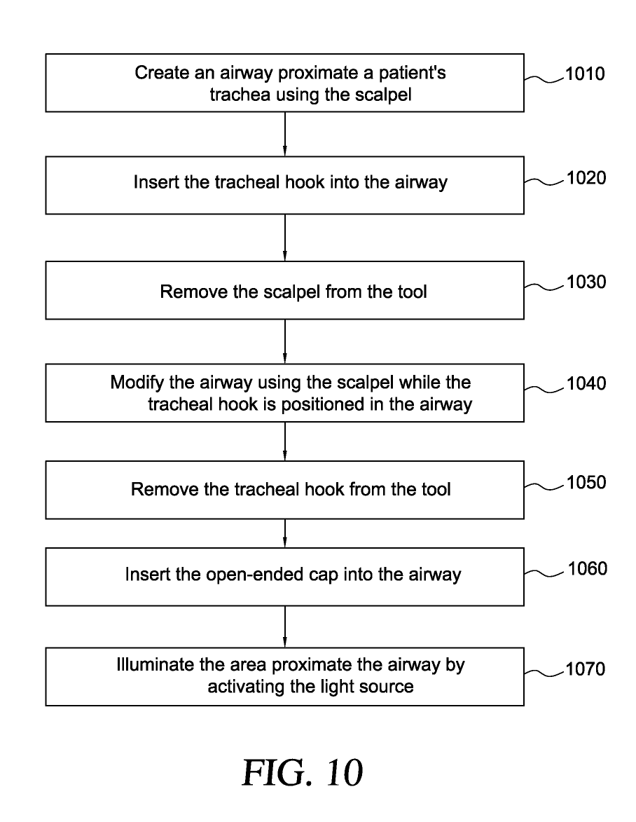

The invention also encompasses a method for utilizing the combination tracheal hook and scalpel tool 100. As illustrated in FIG. 10, an airway is created proximate a patient's trachea using the scalpel 130 of the combination tracheal hook and scalpel tool 100 (1010). The tracheal hook 120 of the combination tracheal hook and scalpel tool 100 is inserted into the airway (1020). The scalpel 130 is removed from the combination tracheal hook and scalpel tool 100 (1030) either before or after the airway is created. The tracheal hook 120 can also be removed from the combination tracheal hook and scalpel tool 100 (1050) either before or after the airway is created. The method as described above is applicable to all embodiments of the combination tracheal hook and scalpel described herein.

In accordance with specific method aspects of the invention, the open-ended cap 700 may be inserted into the airway (1060). As described above, the open-ended cap 700 includes lateral fenestrations 740 to provide additional air passages if one or both of the open ends 710, 720 are blocked by tissue or other obstructions. The area proximate the airway may be illuminated by activating a light source 600 on the combination tracheal hook and scalpel tool 100 (1070). In one embodiment, the hook 120 and/or the handle 110 is formed from transparent or translucent material, such that the light source 600 illuminates the tracheal airway when the hook 120 is inserted into the patient.

We claim:

1. A surgical tool, including:
a handle having a channel;
a tracheal hook connected to said handle; and
a scalpel positioned in said channel of said handle, at least one of said tracheal hook and said scalpel is removably attached to said handle.

2. The surgical tool according to claim 1, wherein said channel includes at least one first fastener and said scalpel includes at least one complementary second fastener for engaging said at least one first fastener.

3. The surgical tool according to claim 2, wherein said at least one first fastener includes a recess, and wherein said at least one complementary second fastener includes a protrusion.

4. The surgical tool according to claim 1, wherein said scalpel includes a light source positioned to illuminate an area proximate said scalpel.

5. The surgical tool according to claim 1, wherein said handle includes a light source positioned to illuminate an area proximate said tracheal hook.

6. The surgical tool according to claim 5, wherein at least one of said tracheal hook and said handle is formed from translucent material.

7. The surgical tool according to claim 1, further including a connector interconnecting said handle and said tracheal hook.

8. The surgical tool according to claim 1, further including at least one cap disposed over at least one of said hook and said scalpel, said at least one cap having a first open end and a second open end.

9. The surgical tool according to claim 7, wherein said cap further includes at least one lateral fenestration.

10. A surgical tool, including:
a tracheal hook having a sheath, said sheath including a channel; and
a scalpel positioned in said channel of said sheath, said scalpel is removably attached to said tracheal hook.

11. The surgical tool according to claim 13, wherein at least one of said handle and said scalpel includes a light source, and wherein at least one of said tracheal hook and said handle is formed from translucent material such that said light source is positioned to illuminate one of said tracheal hook and said handle.

12. A surgical tool, including:
a tracheal hook having an elongated stem including at least one first fastener; and
a scalpel having a handle portion and a blade portion, said handle portion including at least one complementary second fastener for engaging said at least one first fastener,
wherein said scalpel is removably attached to said tracheal hook.

13. The surgical tool according to claim 12, wherein said first fastener includes a T-tab, and wherein said second fastener includes a T-slot.

14. The surgical tool according to claim 12, wherein said first fastener includes a T-slot, and wherein said second fastener includes a T-tab.

15. The surgical tool according to claim 12, wherein at least one of said handle and said scalpel includes a light source, and wherein at least one of said tracheal hook and said handle is formed from translucent material, said light source being positioned to illuminate at least one of said tracheal hook and said handle.

16. A method for utilizing a combination tracheal hook and scalpel tool, said method including:
creating an airway proximate a patient's trachea using a scalpel of said combination tracheal hook and scalpel tool;
inserting a tracheal hook of said combination tracheal hook and scalpel tool into said airway;
removing said scalpel from said combination tracheal hook and scalpel tool; and
modifying said airway using said scalpel while said tracheal hook is positioned in said airway.

17. The method according to claim 16, wherein said removing of said scalpel is performed after said inserting of said tracheal hook.

18. The method according to claim 16, further comprising removing said tracheal hook from said combination tracheal hook and scalpel tool.

19. The method according to claim 16, further comprising illuminating the area proximate said airway by activating a light source on said combination tracheal hook and scalpel tool.

20. The method according to claim 16, further including inserting an open-ended cap into said airway.

21. A surgical tool, including:
a handle having a channel and at least one finger guard, said channel including an enlarged opening having at least one inclined ramp, said at least one finger guard positioned proximate said enlarged opening;
a tracheal hook connected to said handle; and
a scalpel positioned in said channel of said handle, at least one of said tracheal hook and said scalpel is removably attached to said handle.

22. The surgical tool according to claim 21, wherein said handle further includes gripping ridges.
